(19) United States
(12) Patent Application Publication
(10) Pub. No.: US 2026/0007330 A1
(43) Pub. Date: Jan. 8, 2026

(54) ACCELEROMETER-TRIGGERED SENSOR MEASUREMENT ON EXERTION

(71) Applicant: Medtronic, Inc., Minneapolis, MN (US)

(72) Inventors: Geert Morren, Vissenaken (BE); David A. Anderson, Stanchfield, MN (US); Trent M. Fischer, St. Paul, MN (US); Bruce D. Gunderson, Plymouth, MN (US); Shantanu Sarkar, Roseville, MN (US)

(21) Appl. No.: 18/881,510

(22) PCT Filed: Jul. 5, 2023
(86) PCT No.: PCT/IB2023/056973

§ 371 Date: Jan. 6, 2025

Related U.S. Application Data
(60) Provisional application No. 63/369,135, filed on Jul. 22, 2022.

(51) Int. Cl. A61B 5/11; A61B 5/00; G01P 1/07

(52) U.S. Cl. CPC A61B 5/1118; A61B 5/7282; A61B 5/7285; G01P 1/07; A61B 2560/0209; A61B 2562/0219

(57) ABSTRACT

A medical device includes one or more accelerometers; a sensor configured to monitor a physiological parameter of a patient; a memory; and processing circuitry configured to receive, from the one or more accelerometers, an accelerometer signal indicative of an amount of movement of the patient; determine, based on the accelerometer signal, that the patient is in an active period; determine, based on the accelerometer signal, that the active period has ended; and cause the sensor to transition from a low-power state to a high-power state in response to determining that the active period has ended.

This application claims the benefit of U.S. Provisional Patent Application Ser. No. 63/369,135, filed 22 Jul. 2022, the entire content of which is incorporated herein by reference.

### TECHNICAL FIELD

This disclosure generally relates to medical devices and, more particularly, to techniques and devices for monitoring a physiological signal of a patient.

### BACKGROUND

Medical devices may be used to monitor physiological signals of a patient. For example, some medical devices are configured to sense cardiac electrogram (EGM) signals, e.g., electrocardiogram (ECG) signals, indicative of the electrical activity of the heart via electrodes. Those same medical devices or other medical devices may also be configured to sense respiration, pulse oxygen levels, heart rate, or a host of other physiological parameters of a patient.

A computing system may obtain data from medical devices to allow a clinician or other user to review the data acquired for the patient. A clinician may diagnose a medical condition of the patient based on identified occurrences of events associated with the data.

### SUMMARY

To reduce power consumption, it is common for physiological sensors to enter high power states during periods of activity and to transition to low power states when the activity ends. For certain types of physiological sensors, however, such as sensors configured to monitor a signal of interest that is small compared to changes in the measurement that occur due to activity, it can be difficult to measure physiological signals during physical activity, especially physical activity associated with relatively large body motions like walking. At the same time, these physiological signals often provide useful information associated with the time of exertion. Physiological measurements associated with exertion potentially provide additional information compared to measurements taken at rest.

The techniques of this disclosure may improve the diagnostic and monitoring capabilities of a monitoring device by providing reliable physiological data collected during exertion. This disclosure describes techniques to collect reliable sensor data during exertion while also limiting power consumption. According to techniques of this disclosure, a monitoring device may include an accelerometer or other motion or activity sensor integrated into the device. The device can be configured, using the accelerometer, to detect short ‘pauses’ in between the activity or detect the end of the activity. The device may then activate a high-resolution sensor during these ‘pauses’ or right after the end of the activity. For example, upon determining that an active period for the patient has ended and a pause has begun, the device may cause a high-resolution sensor to transition from a low-power state to a high-power state so that the sensor can monitor a physiological parameter of the patient during the pause.

In one example implementation, a monitoring device may be configured to continuously collect accelerometer data indicative of patient movement at a low resolution and based on the accelerometer data, determine that the patient has entered an active period. After the patient has entered the active period, the monitoring device may, based on additional accelerometer data, detect a ‘quiet’ period with little or no motion during or right after the active period. In response to identifying this quiet period, the monitor device may start a high-resolution sensor measurement for a predefined period of time or until the motion, as detected by the accelerometer signal, increases above a certain threshold again for another predefined period of time.

In another example of this disclosure, a medical device includes, one or more accelerometers, a sensor configured to monitor a physiological parameter of a patient, a memory, and processing circuitry configured to: receive, from the one or more accelerometers, an accelerometer signal indicative of an amount of movement of the patient; determine, based on the accelerometer signal, that the patient is in an active period; determine, based on the accelerometer signal, that the active period has ended; and cause the sensor to transition from a low-power state to a high-power state in response to determining that the active period has ended.

In another example of this disclosure, a method includes monitoring, via a sensor of a medical device, a physiological parameter of a patient; receiving, from one or more accelerometers, an accelerometer signal indicative of an amount of movement of the patient; determining, based on the accelerometer signal, that the patient is in an active period; determining, based on the accelerometer signal, that the active period has ended; and causing the sensor to transition from a low-power state to a high-power state in response to determining that the active period has ended.

This summary is intended to provide an overview of the subject matter described in this disclosure. It is not intended to provide an exclusive or exhaustive explanation of the apparatus and methods described in detail within the accompanying drawings and description below. Further details of one or more examples are set forth in the accompanying drawings and the description below.

### BRIEF DESCRIPTION OF DRAWINGS

FIG. 1 is a conceptual drawing illustrating an example of a medical device system configured to monitor a physiological parameter of a patient in accordance with the techniques of the disclosure.

FIG. 2 is a block diagram illustrating an example configuration of the implantable medical device (IMD) of FIG. 1.

FIG. 3 shows an example timing diagram for controlling a sensor in accordance with the techniques of the disclosure.

FIG. 4 is a functional block diagram illustrating an example configuration of the computing system of FIG. 1.

FIG. 5 is a flow diagram illustrating an example operation of accelerometer-triggered sensing in accordance with the techniques of the disclosure.

FIG. 6 is a flow diagram illustrating an example operation of accelerometer-triggered sensing in accordance with the techniques of the disclosure.

Like reference characters refer to like elements throughout the figures and description.

### DETAILED DESCRIPTION

Sensor measurements in medical monitoring devices are often prone to motion artifacts. Artifacts may, for example, occur in cases where a signal of interest, such as a physiological signal, is small compared to changes in the measurement that occur due to motion, i.e., non-physiological artifacts unrelated to the signal of interest. One such example is measuring respiration from subcutaneous (or intrathoracic) impedance in a patient. Changes in subcutaneous impedance due to respiration are on the order of ˜1 Ohm, and changes in impedance due to posture changes or whole-body movement (e.g., walking) can easily be an order of magnitude larger. The large changes in impedance due to movement may be caused by changes in volume of fluid shifts or changes in pressure that occur due to the movement of the patient.

Another example where a signal of interest may be relatively small compared to changes in measurement due to motion is in the case of measuring respiration from an accelerometer signal on the thorax. When measuring respiration from an accelerometer signal on the thorax, the changes in the accelerometer signal, resulting from changes in the tilt angle of the accelerometer due to movement of the chest or ribs caused by respiration, is on the order of ˜20 mg, depending on the location on the thorax and the posture. This is an order of magnitude smaller than the changes observed during other types of upper body movement, such as during walking, where changes of ˜1 g can be observed.

Another example where a signal of interest may be relatively small compared to changes in measurement due to motion is in the case of measuring respiration from the electromyogram (EMG) of the intercostal muscles, or diaphragm, because the EMG signal of the intercostal muscles due to breathing is much smaller than the EMG signal of the intercostal muscle due to other upper body movements. Yet another example is measuring oxygen saturation, blood pressure, or heart rate using an optical sensor, such as a pulse oximeter, because the fluctuations in light intensity due to the arterial pulsatility is much smaller than the changes in light intensity due to whole body movements that cause large shifts of venous blood. A heart rate signal determined from an ECG may also be noisy due to motion or upper body movements.

As evidenced by the examples above, it can be difficult to measure physiological signals during physical activity, especially physical activity associated with relatively large body motions like walking. At the same time, these physiological signals often provide useful information associated with the time of exertion. Physiological measurements associated with exertion potentially provide additional information compared to measurements taken at rest. For example, in the case of respiration, dyspnea (i.e., shortness of breath) is an important symptom of chronic obstructive pulmonary disease (COPD) as well as heart failure and can be used to monitor disease status. Dyspnea is typically most pronounced during and soon after exertion. Dyspnea can be assessed by measuring the EMG of respiratory muscles (intercostal and/or diaphragm) in combination with a measure of (minute) ventilation (e.g., respiration rate x tidal volume) which can be derived from impedance or accelerometer measurements. As another example with respect to oxygen saturation, exercise-induced desaturation is strongly associated with increased mortality and risk of severe exacerbation in COPD patients, whereas patients with exercise-induced desaturation do not necessarily have a low oxygen saturation at rest. As another example with respect to heart rate recovery, the decrease in heart rate at 1 minute after cessation of exercise is a useful prognostic marker in patients with heart failure or coronary artery disease (CAD).

In most wearable, implantable, or insertable monitoring devices, the problem of motion artifacts is addressed using filtering or detection techniques. Even using advanced filtering techniques, however, the signal of interest may not always be reliable during motion. Regardless of the filtering, some monitoring devices have built-in signal quality checks to detect when a signal is reliable and to discard signals that are not reliable due to motion artifacts or other potential artifacts. In either case, the signal is collected and processed continuously during motion artifacts even though the obtained signal may not be reliable or even useable.

Depending on the type and resolution of a sensor, continuous measurements may have a significant impact on current drain, and hence battery longevity, and may also undesirably allocate significant amounts of memory to unusable data. Therefore, this disclosure describes techniques for collecting reliable sensor data during and after a period of exertion in a more efficient way. In this context, a period of exertion may include one or more periods of relatively high activity that are immediately followed periods of low activity. During a period of exertion, the beginning of a period of low activity can be sufficiently close in time to the end of a period of high activity, such that a physiological measurement taken at the beginning of the period of low activity can be considered to be indicative of a state of the patient during the period of high activity.

An accelerometer integrated in the device can be used to detect the end of the activity or short ‘pauses’ during the activity. The device may then activate certain sensors during these ‘pauses’ or right after the end of the activity. In this way, these sensors can be activated during periods of time where the patient's condition is still reflective of the high activity but when the sensors can collect data without the motion artifacts caused by the high activity. Moreover, power consumption can be reduced compared to the continuous collecting and processing of the sensor data. This may be particularly important for high-resolution sensors that consume relatively large amounts power in implantable and insertable devices. Examples of such sensors may include impedance sensors configured to measure respiration, a fluid status, or other such physiological parameters, as well as optical sensors configured to measure heart rate (e.g., based on PPG photoplethysmogram), arterial oxygen saturation (e.g., SpO2, as measured with a pulse oximeter), or other such physiological parameters. Examples of such sensors may also include accelerometer-based sensors for monitoring a patient's posture or gait or acoustic sensors for monitoring heart sounds.

This disclosure describes techniques to collect reliable sensor data during exertion while also limiting power consumption. According to techniques of this disclosure, a monitoring device may include an accelerometer integrated into the device. The device can be configured, using the accelerometer, to detect short ‘pauses’ in between the activity or detect the end of the activity. The device may then activate a high-resolution sensor during these ‘pauses’ or right after the end of the activity. For example, upon determining that an active period for the patient has ended and a pause has begun, the device may cause a high-resolution sensor to transition from a low-power state to a high-power state so that the sensor can monitor a physiological parameter of the patient during the pause.

In one example implementation, a monitoring device may be configured to continuously collect accelerometer data indicative of patient movement at a low resolution and, based on the accelerometer data, determine that the patient has entered an active period. After the patient has entered the active period, the monitoring device may, based on additional accelerometer data, detect a ‘quiet’ period with little or no motion during or right after the active period. In response to identifying this quiet period, the monitoring device may start a high-resolution sensor measurement for a predefined period of time or until the motion, as detected by the accelerometer signal, increases above a certain threshold again for another predefined period of time.

The techniques of this disclosure may improve the diagnostic and monitoring capabilities of a monitoring device by providing reliable physiological data collected during exertion. In many cases, like dyspnea or exercise-induced desaturation, physiological data collection during exertion provides additional prognostic information about the disease status compared to data collected at rest. Furthermore, compared to commonly used techniques to handle motion artefacts, the techniques of this disclosure allow the monitoring device to reduce power consumption and hence increase battery longevity. Battery longevity may be particularly relevant for implantable or insertable devices. The techniques of this disclosure may also be of benefit to memory-limited devices because a monitoring device can be configured to acquire, and store, data related to physiological parameters of a patient during times when such data is likely to have high diagnostic significance and not acquire, or store, such data during times when the data is likely to be corrupted by high amounts of noise.

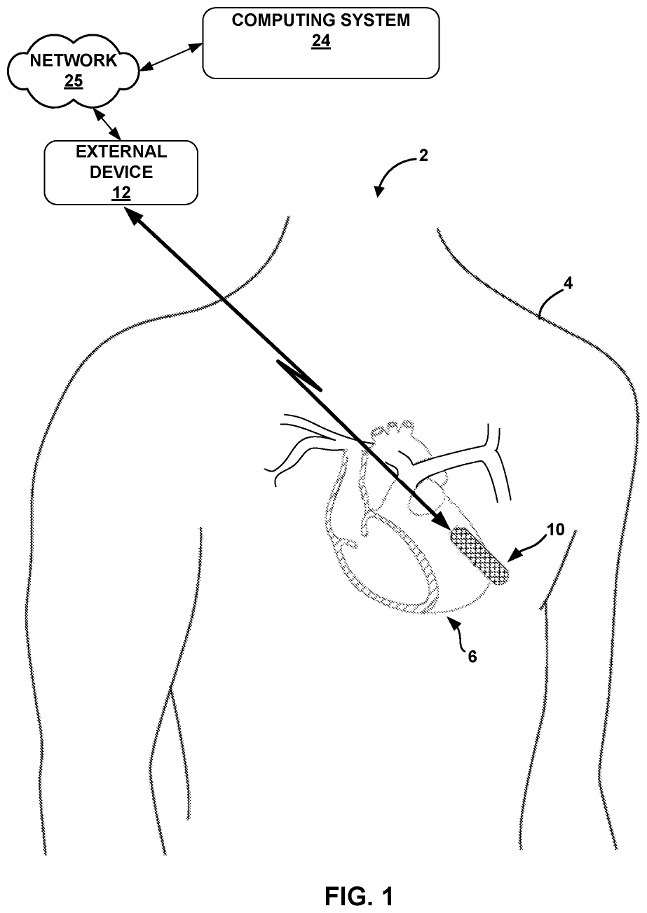

FIG. 1 is a conceptual drawing illustrating an example of a medical device system 2 configured to utilize accelerometer-triggered sensing in accordance with the techniques of the disclosure. The example techniques may be used with an IMD 10, which may be in wireless communication with an external device 12. The techniques of this disclosure will be explained using an implantable cardiac monitoring device, but the described techniques are not limited to such a device. The techniques may, for example, also be implemented in other types of implantable medical devices, such as pacing devices, defibrillation devices, heart or other organ assist devices, as well as insertable and wearable devices, including devices warn on the wrist or arm, fastened to the chest with a chest band, or adhered to the chest or abdomen.

In the example of FIG. 1, IMD 10 is implanted outside of a thoracic cavity of patient 4 (e.g., subcutaneously in the pectoral location illustrated in FIG. 1). IMD 10 may be positioned near the sternum near or just below the level of the heart of patient 4, e.g., at least partially within the cardiac silhouette. IMD 10 includes at least one accelerometer and a plurality of electrodes (not shown in FIG. 1) and is configured to sense a cardiac EGM via the plurality of electrodes. IMD 10 may also include additional sensors, such as optical and impedance sensors. In some examples, IMD 10 takes the form of the LINQ™ ICM. In the context of a cardiac monitor, such as IMD 10 in FIG. 1, the techniques of this disclosure may be of particular benefit for systems configured to detect data indicative of PVCs, PACs, or changes in QT intervals soon after exertion. The techniques of this disclosure may also be of particular benefit for systems configured to detect exertion-induced changes in ECG morphology, such as R-wave amplitude, QRS width, ST shifts, short term HRV, RR interval changes, changes in p-wave amplitude and morphology, etc.

Although the techniques of this disclosure are described primarily in the context of examples in which the medical device that collects episode data takes the form of an ICM, the techniques of this disclosure may be implemented in systems including any one or more implantable or external medical devices, including smart watches, activity monitors, heart rate monitors, blood pressure monitors, Holter monitors, cardiac event monitors and patches, Glucose monitors, pulse oximeters, finger or forehead SpO2 sensors, pacemakers, defibrillators, etc.

IMD 10 may be configured to determine, based on movement detected by an accelerometer, that patient 4 is in an active period and, similarly based on a lack of movement detected by the accelerometer, determine that active period has ended, at least temporarily. In response to determining that the active period has ended, IMD 10 may cause a sensor of IMD 10 to transition from a low-power state to a high-power state, so that the sensor can monitor a physiological parameter of patient 4. The low-power state may, for example, be an off-state or a standby state, and the high-power state may be an on state. In other implementations, in the low-power state, the sensor may monitor the physiological parameter of patient 4 at a first resolution or sampling rate, and in the high-power state, monitor the physiological parameter at a second resolution or sampling rate that is higher than the first sampling rate. The low-power state can reduce the current drain of the sensor relative to the high-power state, and thus improve the battery life of IMD 10.

External device 12 is a computing device configured for wireless communication with IMD 10. External device 12 may be configured to communicate with computing system 24 via network 25. In some examples, external device 12 may provide a user interface and allow a user to interact with IMD 10. Computing system 24 may comprise computing devices configured to allow a user to interact with IMD 10, or data collected from IMD, via network 25.

External device 12 may be used to retrieve data from IMD 10 and may transmit the data to computing system 24 via network 25. The retrieved data may include values of physiological parameters measured by IMD 10, indications of episodes of arrhythmia or other maladies detected by IMD 10, episode data collected for episodes, and other physiological signals recorded by IMD 10. The episode data may include EGM segments recorded by IMD 10, e.g., due to IMD 10 determining that an episode of arrhythmia or another malady occurred during the segment, or in response to a request to record the segment from patient 4 or another user.

In some examples, computing system 24 includes one or more handheld computing devices, computer workstations, servers or other networked computing devices. In some examples, computing system 24 may include one or more devices, including processing circuitry and storage devices. Computing system 24 and network 25 may be implemented, fully or partially, by the Medtronic Carelink™ Network or other patient monitoring systems.

Network 25 may include one or more computing devices (not shown), such as one or more non-edge switches, routers, hubs, gateways, security devices such as firewalls, intrusion detection, and/or intrusion prevention devices, servers, computer terminals, laptops, printers, databases, wireless mobile devices such as cellular phones or personal digital assistants, wireless access points, bridges, cable modems, application accelerators, or other network devices. Network 25 may include one or more networks administered by service providers, and may thus form part of a large-scale public network infrastructure, e.g., the Internet. Network 25 may provide computing devices, such as computing system 24 and IMD 10, access to the Internet, and may provide a communication framework that allows the computing devices to communicate with one another. In some examples, network 25 may be a private network that provides a communication framework that allows computing system 24, IMD 10, and/or external device 12 to communicate with one another but isolates one or more of computing system 24, IMD 10, or external device 12 from devices external to network 25 for security purposes. In some examples, the communications between computing system 24, IMD 10, and external device 12 are encrypted.

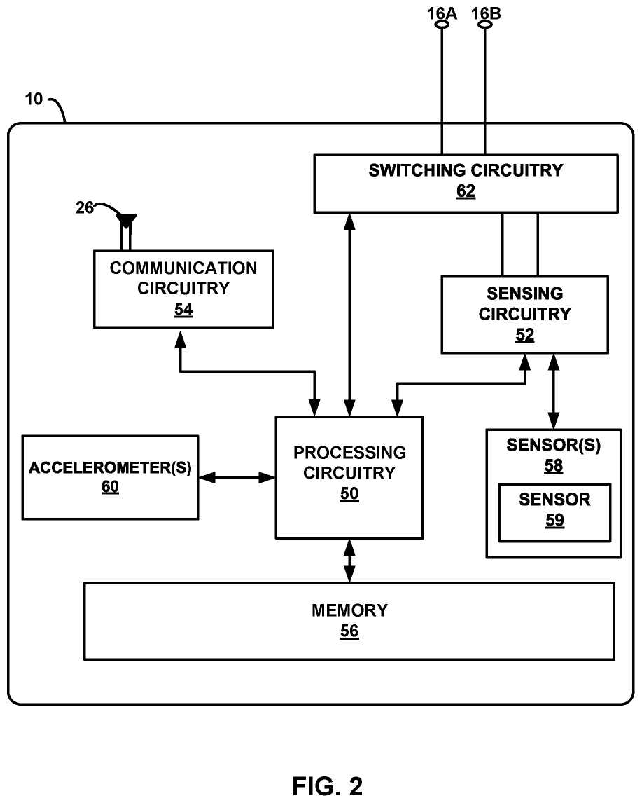

FIG. 2 is a block diagram illustrating an example configuration of IMD 10 of FIG. 1. As shown in FIG. 2, IMD 10 includes processing circuitry 50, sensing circuitry 52, communication circuitry 54, memory 56, sensors 58, accelerometers 60, switching circuitry 62, and electrodes 16A, 16B (hereinafter “electrodes 16”), one or more of which may be disposed on a housing of IMD 10. In some examples, memory 56 includes computer-readable instructions that, when executed by processing circuitry 50, cause IMD 10 and processing circuitry 50 to perform various functions attributed herein to IMD 10 and processing circuitry 50. Memory 56 may include any volatile, non-volatile, magnetic, optical, or electrical media, such as a random-access memory (RAM), read-only memory (ROM), non-volatile RAM (NVRAM), electrically-erasable programmable ROM (EEPROM), flash memory, or any other digital media.

Processing circuitry 50 may include fixed function circuitry and/or programmable processing circuitry. Processing circuitry 50 may include any one or more of a microprocessor, a controller, a digital signal processor (DSP), an application specific integrated circuit (ASIC), a field-programmable gate array (FPGA), or equivalent discrete or analog logic circuitry. In some examples, processing circuitry 50 may include multiple components, such as any combination of one or more microprocessors, one or more controllers, one or more DSPs, one or more ASICs, or one or more FPGAs, as well as other discrete or integrated logic circuitry. The functions attributed to processing circuitry 50 herein may be embodied as software, firmware, hardware or any combination thereof.

Sensing circuitry 52 may be selectively coupled to electrodes 16A, 16B via switching circuitry 62 as controlled by processing circuitry 50. Sensing circuitry 52 may monitor signals from electrodes 16A, 16B in order to monitor electrical activity of a heart of patient 4 of FIG. 1 and produce cardiac EGM data for patient 4. In some examples, processing circuitry 50 may identify features of the sensed cardiac EGM to detect an episode of cardiac arrhythmia of patient 4. Processing circuitry 50 may store the digitized cardiac EGM and features of the EGM used to detect the arrhythmia episode in memory 56 as episode data for the detected arrhythmia episode. In some examples, processing circuitry 50 stores one or more segments of the cardiac EGM data, features derived from the cardiac EGM data, and other episode data in response to instructions from external device 12 (e.g., when patient 4 experiences one or more symptoms of arrhythmia and inputs a command to external device 12 instructing IMD 10 to upload the data for analysis by a monitoring center or clinician).

In some examples, processing circuitry 50 transmits, via communication circuitry 54, the physiological parameter data, as well as other data such as episode data, for patient 4 to an external device, such as external device 12 of FIG. 1. For example, IMD 10 sends values for various physiological parameters, digitized cardiac EGM, and other episode data to network 25 for processing by computing system 24 of FIG. 1.

Sensing circuitry 52 and/or processing circuitry 50 may be configured to detect cardiac depolarizations (e.g., P-waves of atrial depolarizations or R-waves of ventricular depolarizations) when the cardiac EGM amplitude crosses a sensing threshold. For cardiac depolarization detection, sensing circuitry 52 may include a rectifier, filter, amplifier, comparator, and/or analog-to-digital converter, in some examples. In some examples, sensing circuitry 52 may output an indication to processing circuitry 50 in response to sensing of a cardiac depolarization. In this manner, processing circuitry 50 may receive detected cardiac depolarization indicators corresponding to the occurrence of detected R-waves and P-waves in the respective chambers of heart. Processing circuitry 50 may use the indications of detected R-waves and P-waves for determining features of the cardiac EGM including inter-depolarization intervals, heart rate, and detecting arrhythmias, such as tachyarrhythmias and asystole. Sensing circuitry 52 may also provide one or more digitized cardiac EGM signals to processing circuitry 50 for analysis, e.g., for use in cardiac rhythm discrimination and/or to identify and delineate features of the cardiac EGM, such as QRS amplitudes and/or width, or other morphological features.

In some examples, IMD 10 includes one or more sensors 58, such as one or more electrical or biopotential sensors (such as ECG or EMG), chemical sensors, optical sensors, impedance sensors (e.g., skin conductance), temperature sensors, accelerometers (1-axis, 2-axis, and 3-axis), microphones (e.g., acoustic), and/or pressure sensors. Sensors 58 includes sensors 59, which is configured to be controlled by processing circuitry 50 based on detected movement in accordance with the techniques of this disclosure. Sensor 59 may be any one or more of the above-described sensors or be another type of sensors not-described herein. The techniques of this disclosure are not limited to any particular type of sensor. Sensors 58 may also include one or more of additional sensors configured to be controlled based on detected movement, continuously-running sensors, or sensors controlled based on factors other than detected movement.

In some examples, sensing circuitry 52 may include one or more filters and amplifiers for filtering and amplifying signals received from one or more of electrodes 16A, 16B and/or other sensors 58. In some examples, sensing circuitry 52 and/or processing circuitry 50 may include a rectifier, filter and/or amplifier, a sense amplifier, comparator, and/or analog-to-digital converter. Processing circuitry 50 may determine values of physiological parameters of patient 4 based on signals from sensors 58, which may be used to identify arrhythmia episodes and stored as episode data in memory 56.

Sensor 59 may, for example, be a specialized sensor that due to high-power consumption or memory limitations does not run continuously. Sensor 59 may periodically be used in a high-power state by processing circuitry 50 during periods of no activity or low activity to acquire data for purposes of establishing a baseline for patient 4, but generally, the default state for sensor 59 during periods of no activity or low activity may be a low-power state. Additionally or alternatively, sensors 59 may be configured to enter a low-power state when patient 4 enters an active period because sensor 59 is sufficiently affected by noise due to movement of patient 4, such that any data acquired by sensor 59 during an active period of patient 4 is essentially unusable.

Processing circuitry 50 may receive, from accelerometer(s) 60, an accelerometer signal indicative of an amount of movement of patient 4 and determine, based on the accelerometer signal, that patient 4 is in an active period. Accelerometer(s) 60 may include one or more accelerometers, any of which may be 1-axis, 2-axis, or 3-axis accelerometers. During the active period, sensor 59 may be in a low-power state. Upon processing circuitry 50 determining, based on the accelerometer signal received from accelerometer(s) 60, that the active period has ended, processing circuitry 50 may cause sensor 59 to transition from the low-power state to a high-power state.

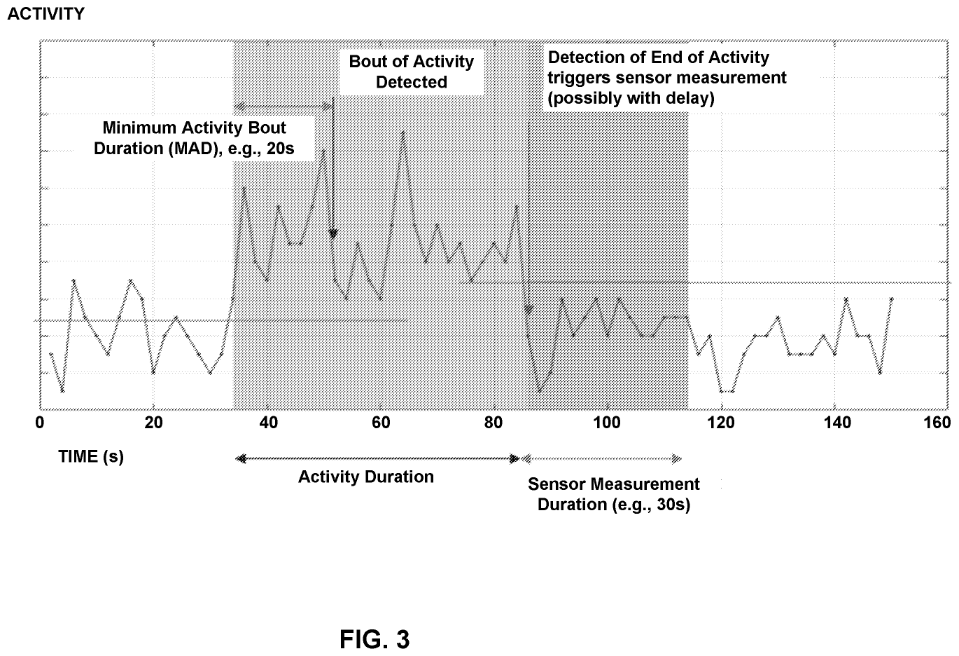

FIG. 3 shows an example timing diagram of how processing circuitry 50 may control sensor 59. In the example of FIG. 3, the x-axis corresponds to time, and the y-axis corresponds to activity. In this context, activity may correspond to a running average or running summation of acceleration-based values determined by accelerometer(s) 60 for a period of time or may correspond to a percentage of time within a period of time for which accelerometer(s) 60 detected acceleration-based values over a certain threshold. Other measurements of activity may also be used.

In the example of FIG. 3, from time 0 until approximately time 35, processing circuitry 50 determines that patient 4 is in a low activity period. At approximately time 35, processing circuitry 50 determines, based on activity, that patient 4 has commenced an active period. At approximately time 85, processing circuitry 50 determines that the active period has ended. From approximately time 35 to 85, sensor 59 may be in a low-power state. Upon determining that the period of activity has ended at approximately time 85, processing circuitry 50 may cause sensor 59 to transition from a low-power state to a high-power state, such that sensor 59 can monitor a physiological parameter of patient 4. After transitioning to the high-power state, sensor 59 may, for example, begin monitoring the physiological parameter or increase alone or both of a resolution or sampling rate at which sensor 59 is monitoring the physiological parameter.

In the example of FIG. 3, sensor 59 monitors the physiological parameter of patient 4 for a duration of 30 seconds, for around time 85 to 115, at which point processing circuitry may cause sensor 59 to transition from the high-power state to a different state. The different state may be the low-power state or a third state distinct from the high-power state or the low-power state. Processing circuitry 50 may cause the sensor to transition from the high-power state to the different state in response to determining that patient 4 has commenced a new active period or based on a timer passing a threshold amount time, such as 30 seconds, 45 seconds, or some other appropriate amount of time. In some examples, processing circuitry 50 may cause sensor 59 to transition from the high-power state to the different state in response to determining that a value of a physiological parameter has passed a threshold value, as the amount of time it takes for patient 4 to return to “normal” may be indicative of patient health. For example, when a respiratory rate of patient 4 has dropped by 30% relative to a peak rate or when a respiratory rate of patient 4 has returned to within 10% of an established baseline rate, then processing circuitry 50 may cause sensor 59 to transition from the high-power state to the different state. In other examples, processing circuitry 50 may cause sensor 59 to transition from the high-power state to the different state in response to determining that an oxygen saturation (SpO2) level has returned to a baseline, such as greater than 95% of a pre-exertion level. In this context, passing a threshold value may mean either being less than or greater than. The amount of time it takes for the patient to return to “normal” may be indicative of patient health.

During the period of activity while sensor 59 is in the high-power state, processing circuitry 50 may store sensor date acquired by sensor 59 to memory 56. Processing circuitry 50 may also store an indication that the sensor data is associated with the active period. In this context, associated with does not necessarily mean during the active period, but may, for example, mean immediately following the active period or in between two active periods. Processing circuitry 50 may also store a metric indicating the amount, duration and/or intensity of activity within a window of time preceding the sensor data being acquired.

Communication circuitry 54 may include any suitable hardware, firmware, software or any combination thereof for communicating with another device, such as external device 12. Under the control of processing circuitry 50, communication circuitry 54 may receive downlink telemetry from, as well as send uplink telemetry to, external device 12 or another device with the aid of an internal or external antenna, e.g., antenna 26. In some examples, processing circuitry 50 may communicate with a networked computing device via an external device (e.g., external device 12) and a computer network, such as the Medtronic CareLink® Network developed by Medtronic, Inc., of Dublin, Ireland.

Although described herein in the context of example IMD 10, the techniques for cardiac arrhythmia detection disclosed herein may be used with other types of devices. For example, the techniques may be implemented with an extra-vascular implantable cardiac defibrillator, a transcatheter pacemaker configured for implantation within the heart, such as the Micra™ transcatheter pacing system commercially available from Medtronic PLC of Dublin Ireland, an insertable cardiac monitor, such as the Reveal LINQ™ ICM, also commercially available from Medtronic PLC, a neurostimulator, a drug delivery device, a medical device external to patient 4, a wearable device such as a wearable cardioverter defibrillator, a fitness tracker, or other wearable device, a mobile device, such as a mobile phone, a “smart” phone, a laptop, a tablet computer, a personal digital assistant (PDA), or “smart” apparel such as “smart” glasses, a “smart” patch, or a “smart” watch.

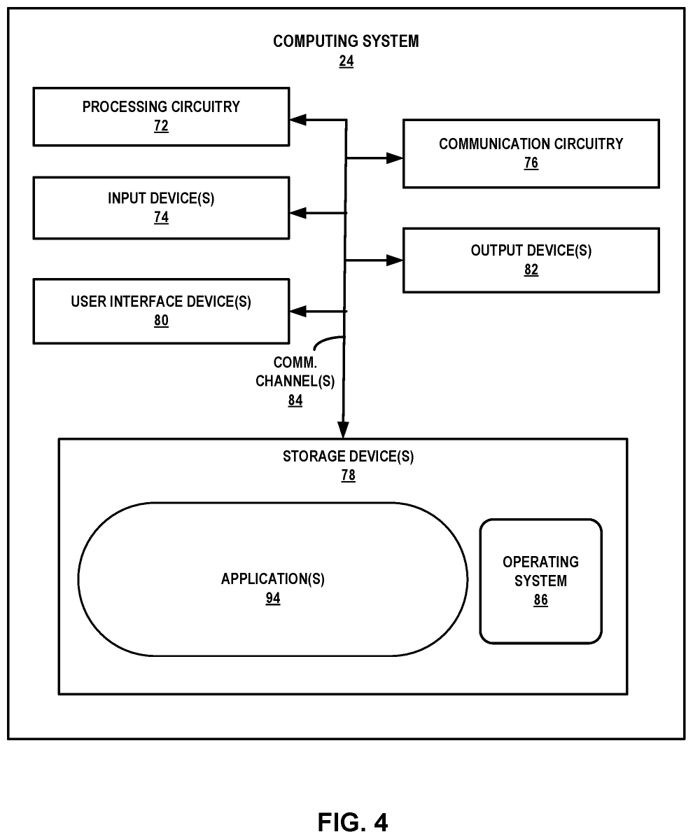

FIG. 4 is a block diagram illustrating an example configuration of computing system 24. In the illustrated example, computing system 24 includes processing circuitry 72 for executing applications 94 which may include cardiac episode monitoring applications or any other applications. Computing system 24 may be any component or system that includes processing circuitry or other suitable computing environment for executing software instructions and, for example, need not necessarily include one or more elements shown in FIG. 4 (e.g., input devices 74, communication circuitry 76, user interface devices 80, or output devices 82; and in some examples components such as storage device(s) 78 may not be co-located or in the same chassis as other components). In some examples, computing system 24 may be a cloud computing system distributed across a plurality of devices.

In the example of FIG. 4, computing system 24 includes processing circuitry 72, one or more input devices 74, communication circuitry 76, one or more storage devices 78, user interface (UI) device(s) 80, and one or more output devices 82. Computing system 24, in some examples, further includes one or more application(s) 94, and operating system 86 that are executable by computing system 24. Each of components 72, 74, 76, 78, 80, and 82 may be coupled (physically, communicatively, and/or operatively) for inter-component communications. In some examples, communication channels 84 may include a system bus, a network connection, an inter-process communication data structure, or any other method for communicating data. As one example, components 72, 74, 76, 78, 80, and 82 may be coupled by one or more communication channels 84.

Processing circuitry 72, in one example, is configured to implement functionality and/or process instructions for execution within computing system 24. For example, processing circuitry 72 may be capable of processing instructions stored in storage device 78. Examples of processing circuitry 72 may include any one or more of a microprocessor, a controller, a digital signal processor (DSP), an application specific integrated circuit (ASIC), a field-programmable gate array (FPGA), or equivalent discrete or integrated logic circuitry.

One or more storage devices 78 may be configured to store information within computing system 24 during operation. Storage device 78, in some examples, is described as a computer-readable storage medium. In some examples, storage device 78 is a temporary memory, meaning that a primary purpose of storage device 78 is not long-term storage. Storage device 78, in some examples, is described as a volatile memory, meaning that storage device 78 does not maintain stored contents when the computer is turned off. Examples of volatile memories include RAM, dynamic random access memories (DRAM), static random access memories (SRAM), and other forms of volatile memories known in the art. In some examples, storage device 78 is used to store program instructions for execution by processing circuitry 72. Storage device 78, in one example, is used by software or applications 94 running on computing system 24 to temporarily store information during program execution.

Storage devices 78, in some examples, also include one or more computer-readable storage media. Storage devices 78 may be configured to store larger amounts of information than volatile memory. Storage devices 78 may further be configured for long-term storage of information. In some examples, storage devices 78 include non-volatile storage elements. Examples of such non-volatile storage elements include magnetic hard discs, optical discs, floppy discs, flash memories, or forms of electrically programmable memories (EPROM) or electrically erasable and programmable memories (EEPROM).

Computing system 24, in some examples, also includes communication circuitry 76 to communicate with other devices and systems, such as IMD 10 and external device 12 of FIG. 1. Communication circuitry 76 may include a network interface card, such as an Ethernet card, an optical transceiver, a radio frequency transceiver, or any other type of device that can send and receive information. Other examples of such network interfaces may include 3G and WiFi radios.

Computing system 24, in one example, also includes one or more user interface devices 80. User interface devices 80, in some examples, are configured to receive input from a user through tactile, audio, or video feedback. Examples of user interface devices(s) 80 include a presence-sensitive display, a mouse, a keyboard, a voice responsive system, video camera, microphone or any other type of device for detecting a command from a user. In some examples, a presence-sensitive display includes a touch-sensitive screen.

One or more output devices 82 may also be included in computing system 24. Output devices 82, in some examples, are configured to provide output to a user using tactile, audio, or video stimuli. Output devices 82, in one example, include a presence-sensitive display, a sound card, a video graphics adapter card, or any other type of device for converting a signal into an appropriate form understandable to humans or machines. Additional examples of output devices 82 include a speaker, a cathode ray tube (CRT) monitor, a liquid crystal display (LCD), or any other type of device that can generate intelligible output to a user.

Computing system 24 may include operating system 86. Operating system 86, in some examples, controls the operation of components of computing system 24. For example, operating system 86, in one example, facilitates the communication of one or more applications 94 with processing circuitry 72, communication circuitry 76, storage device 78, input device 74, user interface devices 80, and output device 82.

Applications 94 may also include program instructions and/or data that are executable by computing system 24. Example application(s) 94 executable by computing system 24 may include cardiac monitoring applications or applications for monitoring other conditions of patient 4. Other additional applications not shown may alternatively or additionally be included to provide other functionality described herein and are not depicted for the sake of simplicity.

In accordance with the techniques of the disclosure, computing system 24 receives episode data for episodes stored by medical devices, such as IMD 10, via communication circuitry 76. Storage device 78 may store the episode data for the episodes in storage device 78. The episode data may have been collected by the medical devices in response to the medical devices detecting arrhythmias and/or user input directing the storage of episode data.

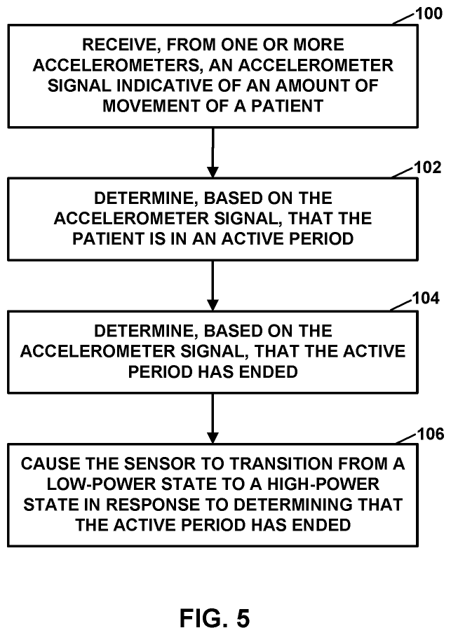

FIG. 5 is a flow diagram illustrating an example operation of accelerometer-triggered sensing in accordance with the techniques of the disclosure. The techniques of FIG. 5 will be described with respect to a generic medical device with a sensor. The medical device may be any of an implantable medical device, an insertable medical device, or a wearable medical device. The sensor may be an optical sensor, an impedance sensor, or some other type of sensor.

According to the example illustrated by FIG. 5, the medical device receives, from one or more accelerometers, an accelerometer signal indicative of an amount of movement of a patient (100).

The medical device determines, based on the accelerometer signal, that the patient is in an active period (102). In some examples, the medical device may cause the sensor to transition from a high-power state to a low-power state in response to determining that the patient is in the active period. In other examples, the sensor may already be in the low-power state prior to sensing that the active period has begun. To determine, based on the signal, that the patient is in the active period, the medical device may be configured to monitor the accelerometer signal for a period of time, determine an amount of time during the period of time for which the user was active, and determine that the patient is in the active period in response to the amount of time being greater than a threshold amount of time. The amount of time during the period of time for which the user was active may correspond to times at which the accelerometer signal indicated that the amount of movement of the patient was above a threshold amount of movement or an amount of time during which the patient had a certain posture, such as an upright posture versus a sitting posture. Additionally or alternatively, to determine, based on the signal, that the patient is in the active period, the medical device may be configured to monitor the accelerometer signal for a period of time, determine a value indicative of a total amount of movement of the patient during the period of time, and determine that the patient is in the active period in response to the value being greater than a threshold value.

The medial device determines, based on the accelerometer signal, that the active period has ended (104). To determine, based on the accelerometer signal, that the active period has ended, the medical device may be configured to monitor the accelerometer signal for a period of time, determine an amount of time during the period of time for which the user was active, and determine that the active period has ended in response to the amount of time being less than a threshold amount of time. The amount of time during the period of time for which the user was active may correspond to times at which the accelerometer signal indicated that the amount of movement of the patient was above a threshold amount of movement. During all or most of the period of time, the sensor may be in the low-power state.

The medical devices causes the sensor to transition from a low-power state to a high-power state in response to determining that the active period has ended (106). The low-power state may, for example, be an off state or a standby state and the high-power state may be an on state. In an off state, the sensor may not perform any sensing or produce a current draw, whereas in a standby state the sensor may not perform any sensing but still produce a relatively small current draw. In some examples, when in the low-power state, the sensor may be configured to still monitor the physiological parameter but monitor the physiological parameter with a sampling rate or resolution that is lower when compared to the high-power state.

In some examples, the medical device may be configured to initiate a timer in response to causing the sensor to transition from the low-power state to the high-power state and cause the sensor to transition from the high-power state to a different state in response to the timer equaling or exceeding a threshold amount time. In some examples, the medical device may monitor the physiological parameter and cause the sensor to transition from the high-power state to a different state in response to a value of the physiological parameter passing a threshold value. In some examples, the medical device may be configured to monitor the accelerometer signal for a second period of time, the second period of time corresponding to a period of time during which the sensor is in the high-power state, determine a second amount of time during the second period of time for which the user was active, determine that the patient is in a second active period in response to the second amount of time being greater than a second threshold amount of time, and cause the sensor to transition from the high-power state to the low-power state in response to determining that the second active period has begun.

The medical device may be configured to store sensor data acquired while the sensor is in the high-power state to the memory. The medical device may also not store sensor data when the sensor is in an off state or a standby state.

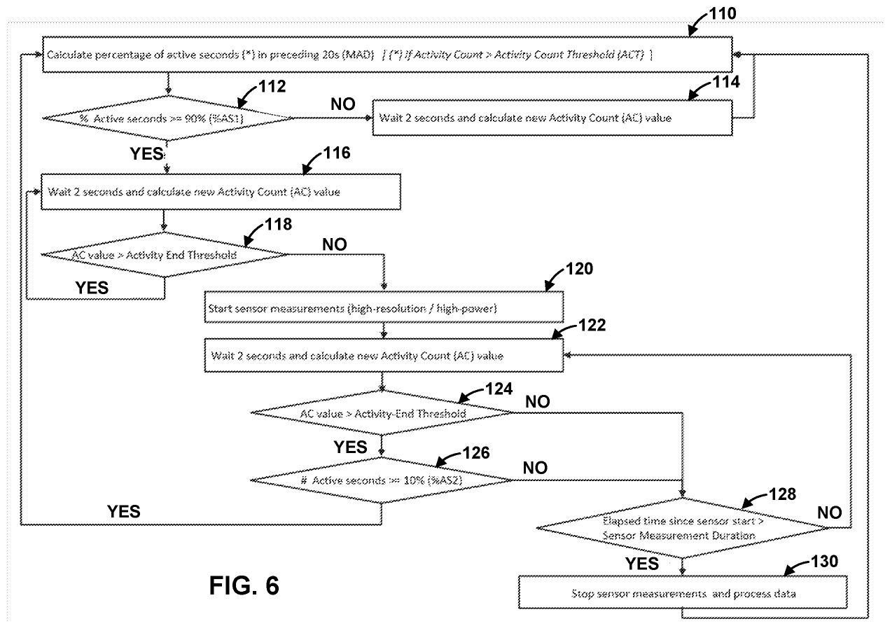

FIG. 6 is a flow diagram illustrating an example operation of accelerometer-triggered sensing in accordance with the techniques of the disclosure. The techniques of FIG. 6 will be described with respect to a generic medical device with a sensor. The medical device may be any of an implantable medical device, an insertable medical device, or a wearable medical device. The sensor may be an optical sensor, an impedance sensor, or some other type of sensor.

According to the example illustrated by FIG. 6, the medical device, e.g., processing circuitry of the medical device, calculates a percentage of active seconds in the preceding 20 seconds during which, based on an accelerometer signal received from one or more accelerometers, a patient was deemed to be active (110). In this context, active seconds may be an amount of time during which a patient is moving a certain amount, but may additionally or alternatively, also correspond to an amount of time during which a patient has a certain posture, such as a standing posture versus a sitting posture. Other periods of time besides 20 seconds may also be used. A patient may be active if an activity count (AC) exceeds an activity count threshold (ACT). If the percentage of active seconds is not greater than or equal to a threshold (112, no), such as 90%, then the medical device has not detected the commencement of an active period and waits two seconds to calculate a new AC value (114). If the percentage of active seconds is greater than or equal to a threshold (112, yes), such as 90%, then the medical device determines that an active period has commenced and waits two seconds to calculate a new AC value (116) to confirm that the active period has continued.

If the AC value is greater than an activity end threshold value (118, yes), then the medical device determines that the patient is still active and continues to calculate new AC values (116). Once the medical device determines that the AC value is less than or equal to the activity end threshold value (118, no), then the medical device determines that the period of activity has ended. Once the period of activity has ended, then the medical device starts obtaining sensor measurements by placing the sensor in a high-power, or high-resolution, mode (120). While the medical device is obtaining the sensor measurements, the medical device may continue to calculate new AC values (122). If the new AC values is greater than the activity end threshold (124, yes), then the medical device may determine if a percentage of active seconds within a period is greater than or equal to a threshold (126). By determining if a percentage of active seconds within a period is greater than or equal to a threshold (126), the medical device may be configured to tolerate small amounts of activity before determining that a new active period has begun. If the percentage of active seconds is greater than or equal to a threshold (126, yes), such as 10%, then the medical device determines that a new active period has commenced, and the medical device begins to again attempt to determine an end to the new active period (110). If the percentage of active seconds is not greater than or equal to the threshold (126, no), then the medical device may determine if an elapsed time since starting the sensor is greater than a sensor measurement duration (128). If the elapsed time since starting the sensor is not greater than the sensor measurement duration (128, no), then the medical device may continue obtaining sensor measurements and continue to determine if patient has commenced a new period of activity (122). If the elapsed time since starting the sensor is greater than the sensor measurement duration (128, yes), then the medical device may stop obtaining sensor measurements and begin processing the data acquired from the sensor measurements (130). Upon stopping obtaining sensor measurements (130), then the medical device may begin to determine if the patient has commenced another active period (110).

In some examples, the techniques of the disclosure include a system that comprises means to perform any method described herein. In some examples, the techniques of the disclosure include a computer-readable medium comprising instructions that cause processing circuitry to perform any method described herein.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module, unit, or circuit for purposes of clarity, it should be understood that the techniques of this disclosure may be performed by a combination of units, modules, or circuitry associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” or “processing circuitry” as used herein may refer to any of the foregoing structure or any other physical structure suitable for implementation of the described techniques. Also, the techniques could be fully implemented in one or more circuits or logic elements.

The following examples are illustrative of the techniques and systems described herein.
Clause 1. A medical device comprising: one or more accelerometers; a sensor configured to monitor a physiological parameter of a patient; a memory; and processing circuitry configured to: receive, from the one or more accelerometers, an accelerometer signal indicative of an amount of movement of the patient; determine, based on the accelerometer signal, that the patient is in an active period; determine, based on the accelerometer signal, that the active period has ended; and cause the sensor to transition from a low-power state to a high-power state in response to determining that the active period has ended.
Clause 2. The medical device of clause 1, wherein the processing circuitry is further configured to: cause the sensor to transition from the high-power state to the low-power state in response to determining that the patient is in the active period.
Clause 3. The medical device of clause 1 or 2, wherein to determine, based on the accelerometer signal, that the patient is in the active period, the processing circuitry is further configured to: monitor the accelerometer signal for a period of time; determine an activity count during the period of time; and determine that the patient is in the active period in response to the activity count being greater than a threshold amount of time.
Clause 4. The medical device of clause 3, wherein the activity count corresponds to times at which the accelerometer signal indicated that the amount of movement of the patient was above a threshold amount of movement.
Clause 5. The medical device of clause 1 or 2, wherein to determine, based on the accelerometer signal, that the patient is in the active period, the processing circuitry is further configured to: monitor the accelerometer signal for a period of time; determine a value indicative of a total amount of movement of the patient during the period of time; and determine that the patient is in the active period in response to the value being greater than a threshold value.
Clause 6. The medical device of clause 1 or 2, wherein to determine, based on the accelerometer signal, that the active period has ended, the processing circuitry is configured to: monitor the accelerometer signal for a period of time; determine an activity count during the period of time for which the user was active; and determine that the active period has ended in response to the activity count being less than an activity count threshold.
Clause 7. The medical device of clause 6, wherein the activity count corresponds to times at which the accelerometer signal indicated that the amount of movement of the patient was above a threshold amount of movement.
Clause 8. The medical device of clause 6, wherein the period of time comprises a period of time during which the sensor is in the low-power state.
Clause 9. The medical device of any of clauses 1-8, wherein the processing circuitry is further configured to: initiate a timer in response to causing the sensor to transition from the low-power state to the high-power state; and cause the sensor to transition from the high-power state to a different state in response to the timer equaling or exceeding a threshold amount time.
Clause 10. The medical device of any of clauses 1-9, wherein the processing circuitry is further configured to: monitor the physiological parameter; and cause the sensor to transition from the high-power state to a different state in response to a value of the physiological parameter passing a threshold value.
Clause 11. The medical device of any of clauses 1-10, wherein the processing circuitry is further configured to: monitor the accelerometer signal for a second period of time, the second period of time corresponding to a period of time during which the sensor is in the high-power state; determine a second amount of time during the second period of time for which the user was active; determine that the patient is in a second active period in response to the second amount of time being greater than a second threshold amount of time; and cause the sensor to transition from the high-power state to the low-power state in response to determining that the second active period has begun.
Clause 12. The medical device of any of clauses 1-11, wherein the low-power state comprises an off state and the high power state comprises an on state.
Clause 13. The medical device of any of clauses 1-12, wherein the low-power state comprises a standby state and the high power state comprises an on state.
Clause 14. The medical device of any of clauses 1-13, wherein: in the low-power state, the sensor monitors the physiological parameter of the patient at a first sampling rate, and in the high-power state, the sensor monitors the physiological parameter of the patient at a second sampling rate that is higher than the first sampling rate.
Clause 15. The medical device of any of clauses 1-14, wherein: in the low-power state, the sensor monitors the physiological parameter of the patient at a first sampling resolution, and in the high-power state, the sensor monitors the physiological parameter of the patient at a second sampling resolution that is higher than the first sampling resolution.
Clause 16. The medical device of any of clauses 1-15, wherein the sensor comprises an optical sensor.
Clause 17. The medical device of any of clauses 1-15, wherein the sensor comprises an impedance sensor.
Clause 18. The medical device of any of clauses 1-17, wherein the processing circuitry is further configured to: store sensor data acquired while the sensor is in the high-power state to the memory.
Clause 19. The medical device of any of clauses 1-18, wherein the medical device comprises an implantable medical device.
Clause 20. The medical device of any of clauses 1-18, wherein the medical device comprises a wearable medical device.
Clause 21. A method comprising: monitoring, via a sensor of a medical device, a physiological parameter of a patient; receiving, from one or more accelerometers, an accelerometer signal indicative of an amount of movement of the patient; determining, based on the accelerometer signal, that the patient is in an active period; determining, based on the accelerometer signal, that the active period has ended; and causing the sensor to transition from a low-power state to a high-power state in response to determining that the active period has ended.
Clause 22. The method of clause 21, further comprising: causing the sensor to transition from the high-power state to the low-power state in response to determining that the patient is in the active period.
Clause 23. The method of clause 21 or 22, wherein determining, based on the accelerometer signal, that the patient is in the active period comprises: monitoring the accelerometer signal for a period of time; determining an activity count during the period of time; and determining that the patient is in the active period in response to the activity count being greater than a threshold amount of time.
Clause 24. The method of clause 23, wherein the activity count corresponds to times at which the accelerometer signal indicated that the amount of movement of the patient was above a threshold amount of movement.
Clause 25. The method of clause 21 or 22, wherein determining, based on the accelerometer signal, that the patient is in the active period comprises: monitoring the accelerometer signal for a period of time; determining a value indicative of a total amount of movement of the patient during the period of time; and determining that the patient is in the active period in response to the value being greater than a threshold value.
Clause 26. The method of clause 21 or 22, wherein determining, based on the accelerometer signal, that the active period has ended comprises: monitoring the accelerometer signal for a period of time; determining an amount of time during the period of time for which the user was active; and determining that the active period has ended in response to the amount of time being less than a threshold amount of time.
Clause 27. The method of clause 26, wherein the activity count corresponds to times at which the accelerometer signal indicated that the amount of movement of the patient was above a threshold amount of movement.
Clause 28. The method of clause 26, wherein the period of time comprises a period of time during which the sensor is in the low-power state.
Clause 29. The method of any of clauses 21-28, further comprising: initiating a timer in response to causing the sensor to transition from the low-power state to the high-power state; and causing the sensor to transition from the high-power state to a different state in response to the timer equaling or exceeding a threshold amount time.
Clause 30. The method of any of clauses 21-29, further comprising: monitoring the physiological parameter; and causing the sensor to transition from the high-power state to a different state in response to a value of the physiological parameter passing a threshold value.
Clause 31. The method of any of clauses 21-30, further comprising: monitoring the accelerometer signal for a second period of time, the second period of time corresponding to a period of time during which the sensor is in the high-power state; determining a second amount of time during the second period of time for which the user was active; determining that the patient is in a second active period in response to the second amount of time being greater than a second threshold amount of time; and causing the sensor to transition from the high-power state to the low-power state in response to determining that the second active period has begun.
Clause 32. The method of any of clauses 21-31, wherein the low-power state comprises one of an off state or a standby state and the high power state comprises an on state.
Clause 33. The method of any of clauses 21-32, wherein: in the low-power state, the sensor monitors the physiological parameter of the patient at a first sampling rate, and in the high-power state, the sensor monitors the physiological parameter of the patient at a second sampling rate that is higher than the first sampling rate.
Clause 34. The method of any of clauses 21-33, wherein: in the low-power state, the sensor monitors the physiological parameter of the patient at a first sampling resolution, and in the high-power state, the sensor monitors the physiological parameter of the patient at a second sampling resolution that is higher than the first sampling resolution.
Clause 35. The method of any of clauses 21-34, wherein the sensor comprises one of an optical sensor or an impedance sensor.
Clause 36. The method of any of clauses 21-34, further comprising: storing sensor data acquired while the sensor is in the high-power state to the memory.
Clause 37. The method of any of clauses 21-36, wherein the medical device comprises one of an implantable medical device or a wearable medical device.

Various examples have been described. These and other examples are within the scope of the following claims.

1. A medical device comprising:
one or more accelerometers;
a sensor configured to monitor a physiological parameter of a patient;
a memory; and
processing circuitry configured to:
receive, from the one or more accelerometers, an accelerometer signal indicative of an amount of movement of the patient;
while the sensor is in a low-power state, determine, based on the accelerometer signal, that the patient has entered an active period;
while the sensor is in the low-power state, determine, based on the accelerometer signal, that the active period has ended; and
cause the sensor to transition from the low-power state to a high-power state in response to determining the patient has entered the active period and determining that the active period has ended.

2. The medical device of claim 1, wherein the processing circuitry is further configured to:
cause the sensor to transition from the high-power state to the low-power state in response to determining that the patient is in the active period.

3. The medical device of claim 1, wherein to determine, based on the accelerometer signal, that the patient has entered the active period, the processing circuitry is further configured to:
monitor the accelerometer signal for a period of time;
determine an activity count during the period of time; and
determine that the patient has entered the active period in response to the activity count being greater than a threshold amount of time.

4. The medical device of claim 3, wherein the activity count corresponds to times at which the accelerometer signal indicated that the amount of movement of the patient was above a threshold amount of movement.

5. The medical device of claim 1, wherein to determine, based on the accelerometer signal, that the patient has entered the active period, the processing circuitry is further configured to:
monitor the accelerometer signal for a period of time;
determine a value indicative of a total amount of movement of the patient during the period of time; and
determine that the patient has entered the active period in response to the value being greater than a threshold value.

6. The medical device of claim 1, wherein to determine, based on the accelerometer signal, that the active period has ended, the processing circuitry is configured to:
monitor the accelerometer signal for a period of time;
determine an activity count during the period of time for which the patient was active; and
determine that the active period has ended in response to the activity count being less than an activity count threshold.

7. The medical device of claim 6, wherein the activity count corresponds to times at which the accelerometer signal indicated that the amount of movement of the patient was above a threshold amount of movement.

8. The medical device of claim 6, wherein the period of time comprises a period of time during which the sensor is in the low-power state.

9. The medical device of claim 6, wherein the activity count threshold is equal to ninety percent of the period of time.

10. The medical device of claim 1, wherein the processing circuitry is further configured to:
initiate a timer in response to causing the sensor to transition from the low-power state to the high-power state; and
cause the sensor to transition from the high-power state to a different state in response to the timer equaling or exceeding a threshold amount time.

11. The medical device of claim 10, wherein the threshold amount of time is less than 45 seconds.

12. The medical device of claim 1, wherein the processing circuitry is further configured to:
monitor the physiological parameter; and
cause the sensor to transition from the high-power state to a different state in response to a value of the physiological parameter passing a threshold value.

13. The medical device of claim 1, wherein the processing circuitry is further configured to:
monitor the accelerometer signal for a second period of time, the second period of time corresponding to a period of time during which the sensor is in the high-power state;
determine a second amount of time during the second period of time for which the patient was active;
determine that the patient is in a second active period in response to the second amount of time being greater than a second threshold amount of time; and
cause the sensor to transition from the high-power state to the low-power state in response to determining that the second active period has begun.

14. The medical device of claim 1, wherein the low-power state comprises an off state and the high power state comprises an on state.

15. A method comprising:
monitoring, via a sensor of a medical device, a physiological parameter of a patient;
receiving, from one or more accelerometers, an accelerometer signal indicative of an amount of movement of the patient;
while the sensor is in a low-power state, determining, based on the accelerometer signal, that the patient has entered an active period;
while the sensor is in the low-power state, determining, based on the accelerometer signal, that the active period has ended; and
causing the sensor to transition from the low-power state to a high-power state in response to determining the patient has entered the active period and determining that the active period has ended.

16. The method of claim 15, further comprising:
causing the sensor to transition from the high-power state to the low-power state in response to determining that the patient is in the active period.

17. The method of claim 15, further comprising:
initiating a timer in response to causing the sensor to transition from the low-power state to the high-power state; and
causing the sensor to transition from the high-power state to a different state in response to the timer equaling or exceeding a threshold amount time.

18. The method of claim 15, further comprising:
causing the sensor to transition from the high-power state to a different state in response to a value of the physiological parameter passing a threshold value.

19. The method of claim 15, further comprising:
monitoring the accelerometer signal for a second period of time, the second period of time corresponding to a period of time during which the sensor is in the high-power state;
determining a second amount of time during the second period of time for which the patient was active;
determining that the patient is in a second active period in response to the second amount of time being greater than a second threshold amount of time; and
causing the sensor to transition from the high-power state to the low-power state in response to determining that the second active period has begun.

20. The method of claim 15, wherein the low-power state comprises an off state and the high power state comprises an on state.
